(19) United States
(12) Patent Application Publication
(10) Pub. No.: US 2005/0175858 A1
(43) Pub. Date: Aug. 11, 2005

(54) Carbazole containing compound and organic electroluminescent display device using the same

(76) Inventors: Dong-Hyun Jung, Suwon-si (KR); Soo-Jin Park, Seoul (KR); Dae-Yup Shin, Suwon-si (KR); Joon-Young Park, Seoul (KR)

Correspondence Address: MCGUIREWOODS, LLP, 1750 TYSONS BLVD, SUITE 1800, MCLEAN, VA, 22102, US

(21) Appl. No.: 10/976,231

(22) Filed: Oct. 29, 2004

(30) Foreign Application Priority Data
Oct. 29, 2003 (KR) 2003-75803

(51) Int. Cl. H05B 33/14; C07D 209/82

(52) U.S. Cl. 428/690; 428/917; 313/504; 313/506; 548/440

(57) ABSTRACT

The present invention is directed to a carbazole containing compound and an organic electroluminescent display device using the same. In particular, the carbazole containing compound may be a blue phosphorescent host material having a large triplet energy gap compared to 4,4′-N,N′-dicarbazole-biphenyl (CBP) and effectively transfers energy to a blue dopant. An organic layer formed using the carbazole containing compound may be used in an organic electroluminescent display device in order to provide high efficiency, improved color purity, and improved power consumption characteristics.

### CROSS-REFERENCE TO RELATED APPLICATIONS

This application claims priority to Korean Patent Application No. 2003-75803, filed on Oct. 29, 2003, in the Korean Intellectual Property Office, the disclosure of which is incorporated herein in its entirety by reference.

### FIELD OF THE INVENTION

The present invention is related to a carbazole containing compound and an organic electroluminescent display device using the same. In particular, the carbazole compound of the present invention may be used as the blue light emitting phosphorescent host.

### BACKGROUND

Organic electroluminescent display device (EL device) light emitting materials are divided into fluorescent materials and phosphorescent materials. Typically, fluorescent materials use exitons in a singlet state and phosphorescent materials use exitons in a triplet state, depending on its light emitting mechanism. In general, phosphorescent materials have an organometallic compound structure containing heavy atoms and emit light via an allowed transition. A phosphorescent material using triplet exitons having a generating probability of 75% has higher luminescence efficiency than a fluorescent material using singlet exitons having a generating probability of 25%.

A phosphorescent light emitting layer is composed of a host material and a dopant material. The dopant material receives energy from the host material resulting in light emission. The dopant materials may include compounds such as iridium metal compounds. Specifically, (4,6-F₂ppy)₂Irpic (Adachi et al., 79 APPL. PHYS. LETT., 3082-3084 (2001)) and Ir compounds based on a fluorinated ppy ligand structure (Grushin et al., CHEM. COMMUN., 1494-1495 (2001)) have been developed as a blue light emitting material, and 4,4′-N,N′-dicarbazole-biphenyl (CBP) materials have been widely used as host materials. The triplet band gap of CBP is sufficient to produce green and red light, but is too small to produce blue light as an exothermic energy transition. Since the CBP host does not exothermically produce blue light, problems such as low blue light emitting efficiency and a shorter lifetime may occur.

mCP (1,3-bis(carbazol-9-yl)-benzene) compounds having a larger triplet energy band gap than CBP compounds have been used, but have very low molecular weight and low stability. Thus, in order to obtain high efficiency and long lifetime for a blue light emitting material, a host material having a larger triplet energy band gap than CBP may be required to efficiently transfer energy to a blue dopant.

### SUMMARY OF THE INVENTION

The present invention is related to a blue light emitting compound having a larger triplet energy band gap than 4,4′-N,N′-dicarbazole-biphenyl (CBP) in order to efficiently transfer energy to a blue dopant.

Another aspect of the present invention is directed to an organic EL device having an organic layer composed of a blue light emitting compound having a larger triplet energy band gap than 4,4′-N,N′-dicarbazole-biphenyl (CBP). As a result, the EL device will have improved color purity, improved power consumption characteristics, and improved luminescence efficiency.

According to an aspect of the present invention, a carbazole containing compound of Formula (1) is provided below: 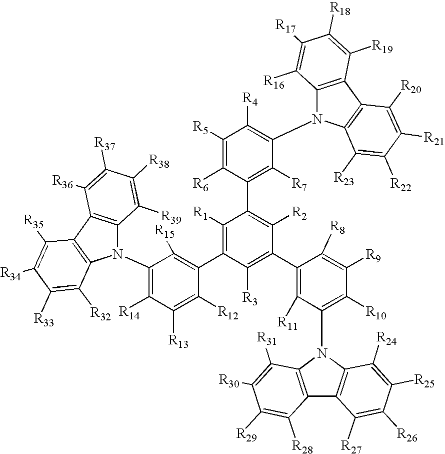
where each of R₁ through R₃₉ may independently be a hydrogen atom, a cyano group, a hydroxy group, a thiol group, a halogen atom, a substituted or unsubstituted C1-C30 alkyl group, a substituted or unsubstituted C1-C30 alkoxy group, a substituted or unsubstituted C2-C30 alkenyl group, a substituted or unsubstituted C2-C30 alkylcarbonyl group, a substituted or unsubstituted C6-C30 aryl group, a substituted or unsubstituted C7-C30 arylcarbonyl group, a substituted or unsubstituted C6-C30 arylalkyl group, a substituted or unsubstituted C6-C30 aryloxy group, a substituted or unsubstituted C2-C30 heteroaryl group, a substituted or unsubstituted C2-C30 heteroarylalkyl group, a substituted or unsubstituted C2-C30 heteroaryloxy group, a substituted or unsubstituted C2-C30 heteroarylalkyloxy group, a substituted or unsubstituted C5-C30 cycloalkyl group, a substituted or unsubstituted C2-C30 heterocycloalkyl group, a substituted or unsubstituted C1-C30 alkylthio group, —Si(R′)(R″)(R′″) (where each of R′, R″, and R′″ is hydrogen or a C1-C30 alkyl group) and —N(R′)(R″) (where each of R′ and R″ is hydrogen or a C1-C30 alkyl group). Alternatively, each of R₁ through R₃₉ may independently be two or more adjacent groups including R₄-R₆, R₈-R₁₀, R₁₂-R₁₄, R₁₆-R₁₉, R₂₀-R₂₃, R₂₄-R₂₇, R₂₈-R₃₁, R₃₂-R₃₅, and R₃₆-R₃₉ that are connected to each other and may form an unsaturated or saturated ring.

According to another aspect of the present invention, there is provided an organic EL device comprising an organic layer interposed between a pair of electrodes, where the organic layer comprises the above-described carbazole containing compound.

### BRIEF DESCRIPTION OF THE DRAWINGS

The above and other features and advantages of the present invention will become more apparent by describing in detail exemplary embodiments thereof with reference to the attached drawings.

FIG. 1 is a cross-sectional view of a general organic EL device.

FIG. 2 is ¹H-NMR spectrum of a compound of Formula (2).

FIG. 3 is photoluminescence (PL) spectrum of a compound of Formula (2) at room temperature and at 77K.

FIG. 4 is PL spectrum of the compound of Formula (2) in a CH₂Cl₂ solution.

FIG. 5 is a solid PL spectrum of a thin film prepared by doping polymethylmethacrylate (PMMA) polymer with the compound of Formula (2).

FIG. 6 is a solid PL spectrum of a thin film prepared by coating PMMA polymer with the compound of Formula (2) doped with EB002 dopant.

FIG. 7 is a graph showing current density with respect to voltage of an organic EL device prepared according to Example 1.

FIG. 8 is a graph showing efficiency with respect to luminance of an organic EL device prepared according to Example 1.

### DETAILED DESCRIPTION OF THE INVENTION

The present invention is directed to a carbazole containing compound of Formula (1) as described above. The structure of Formula (1) may be obtained by introducing carbazoles into the third carbon position (meta position) of the phenyl (A) group of a triphenylbenzene structure. This structure may result in a larger triplet energy band gap than when carbazoles are introduced into para positions of a triphenylbenzene structure. Since the molecular weight of the carbazole containing compound is 200 times larger than CBP, it can have both a higher glass transition temperature Tg and an increased thin film stability.

In a specific embodiment, the compound of Formula (1) may be used as the host in an emissive layer. Moreover, the host may have a larger triplet energy band gap than the blue dopant. Hence, this will effectively allow transfer of the energy from the host to the dopant, thereby resulting in higher luminescence efficiency.

Further, in an organic EL device according to an embodiment of the present invention, the compound of Formula (1) may be used to form an emissive layer. In an alternative embodiment, the compound of Formula (1) may be used as a host in an emissive layer to form an organic layer. The organic EL device may have higher luminescence efficiency than a conventional blue light emitting compound, thereby resulting in reduced power consumption.

In Formula (1), as described above, two or more of adjacent groups among R₄-R₆, R₈-R₁₀, R₁₂-R₁₄, R₁₆-R₁₉, R₂₀-R₂₃, R₂₄-R₂₇, R₂₈-R₃₁, R₃₂-R₃₅, and R₃₆-R₃₉ may be linked together to form an unsaturated or saturated ring. The unsaturated or saturated ring may be C2-C30 carbon cycle or heterocycle. The cycle may be attached or fused together by a pendent method.

Specific examples of the carbazole containing compound of Formula (1) include, but are not limited to, a compound of Formula (1) where each of R₆, R₈, and R₁₂ is a methoxy group, and where each of R₁-R₅, R₇, R₉, R₁₀, R₁₁, and R₁₃-R₃₉ is hydrogen, thereby forming the compound of Formula (2), infra, a compound of Formula (1) where each of R₆, R₈, and R₁₂ is a methyl group, and where each of R₁-R₅, R₇, R₉, R₁₀, R₁₁, and R₁₃-R₃₉ is hydrogen, forming the compound of Formula (3),infra; a compound of Formula (1) where each of R₄, R₇, R₁₀, R₁₁, R₁₄, and R₁₅ is a methyl group, and where each of R₁-R₃, R₅, R₆, R₈, R₉, R₁₂, R₁₃ and R₁₆-R₃₉ is hydrogen, forming the compound of Formula (4), as decribed below; the compound of Formula (1) where each of R₄, R₆, R₈, R₁₀, R₁₂, and R₁₄ is a methyl group, and where each of R₁-R₃, R₅, R₇, R₉, R₁₁, R₁₃, and R₁₅-R₃₉ is hydrogen, that forms a compound of Formula (5), as described below; a compound of Formula (1) where each of R₁-R₃₉ is hydrogen, that form the compound of Formula (6), infra; a compound of Formula (1) where each of R₄, R₆, R₈, R₁₀, R₁₂, R₁₄, R₁₈, R₂₁, R₂₆, R₂₉, R₃₄, and R₃₇ is a methyl group, and where each of R₁-R₃, R₅, R₇, R₉, R₁₁, R₁₃, R₁₅-R₁₇, R₁₉, R₂₀, R₂₂-R₂₅, R₂₇, R₂₈, R₃₀-R₃₃, R₃₅, R₃₆, R₃₈, and R₃₉ is hydrogen, forming the compound of Formula (7), as descibed below; and a compound of Formula (8), infra:

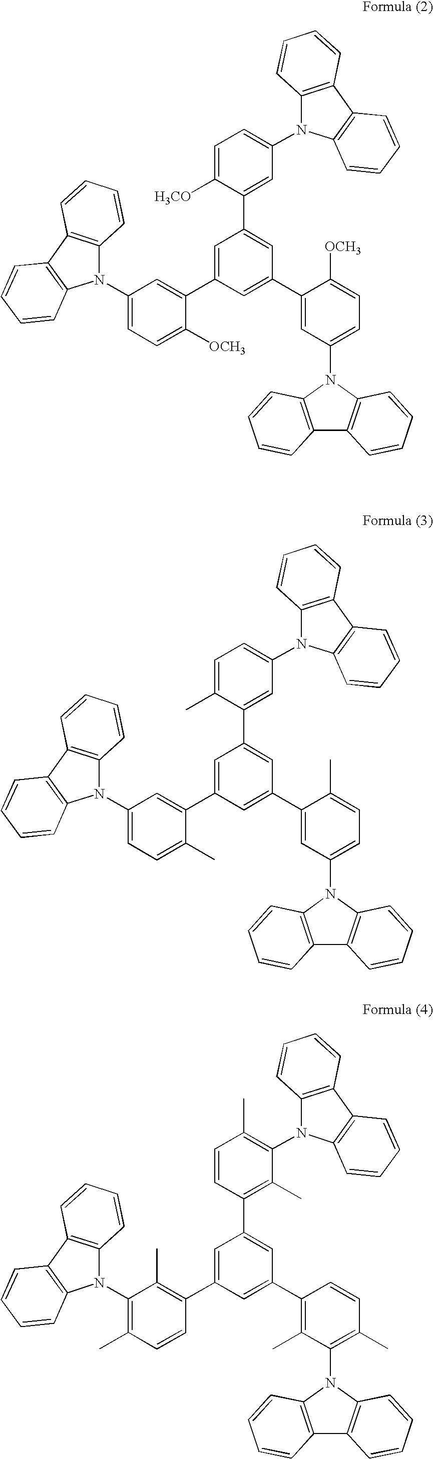

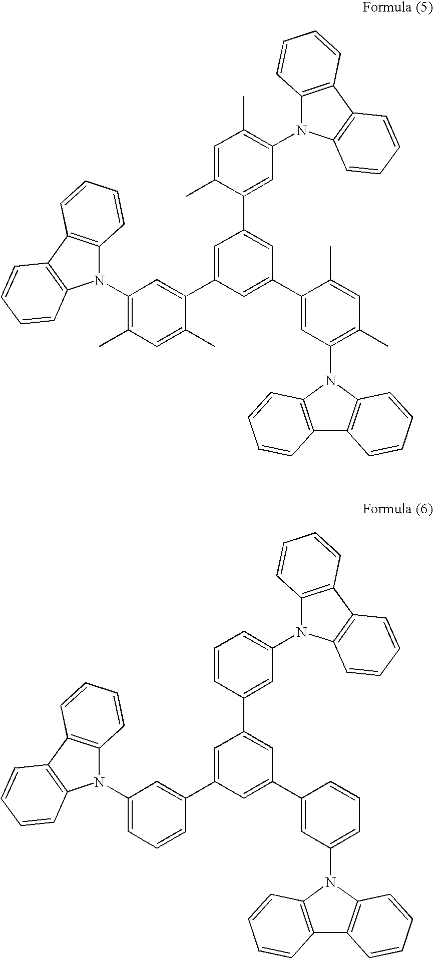

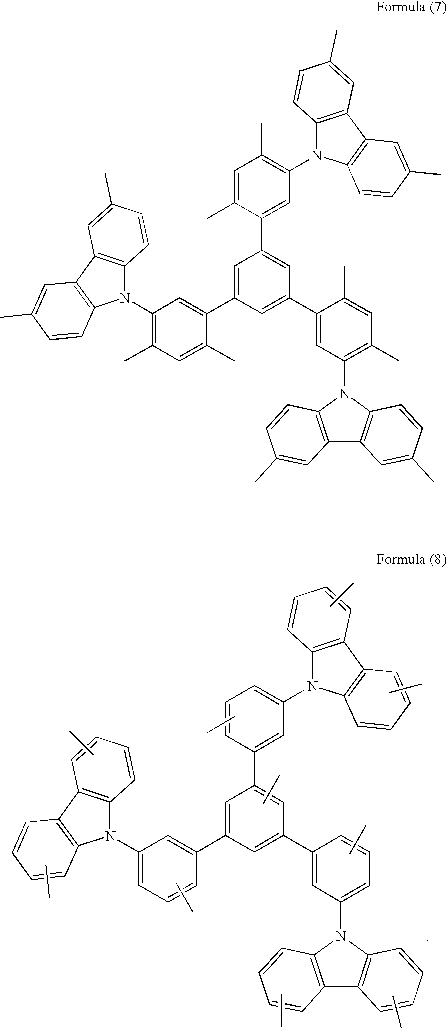

In one embodiment of the present invention, specific examples of the unsubstituted C1-C30 alkyl group in Formula (1) may include, but are not limited to, methyl, ethyl, propryl, isopropryl, sec-butyl, pentyl, iso-amyl, hexyl, and the like. Furthermore, at least one hydrogen atom of the alkyl group may be substituted with a halogen atom, a hydroxy group, a nitro group, a cyano group, an amino group, an amidino group, hydrazine, hydrazone, a carboxyl group or a salt thereof, a sulfonic acid group or a salt thereof, a phosphoric acid group or a salt thereof, a C1-C30 alkyl group, a C1-C30 alkenyl group, a C1-C30 alkynyl group, a C6-C30 aryl group, a C7-C30 arylalkyl group, C2-C30 heteroaryl group, or a C3-C30 heteroarylalkyl group.

In another embodiment, the unsubstituted C2-C30 alkylcarbonyl group in Formula (1) may include acetyl, ethylcarbonyl, isoproprylcarbonyl, phenylcarbonyl, naphthalenecarbonyl, diphenylcarbonyl, cyclohexylcarbonyl, and the like, for example. Moreover, at least one hydrogen atom of the alkylcarbonyl group may be substituted with the same substituent as in the above-described alkyl group.

In a further embodiment, specific examples of the unsubstituted C7-C30 arylcarbonyl group in Formula (1) may include, but is not limited to, phenylcarbonyl, naphthalenecarbonyl, diphenylcarbonyl, and the like. Additionally, at least one hydrogen atom of the arylcarbonyl group may be substituted with the same substituent as in the above-described alkyl group.

In another embodiment, specific examples of the unsubstituted C1-C30 alkoxy group in Formula (1) may include methoxy, ethoxy, phenyloxy, cyclohexyloxy, naphthyloxy, isopropyloxy, diphenyloxy, and the like. Furthermore, at least one hydrogen atom of the alkoxy group may be substituted with the same substituent as in the above-described alkyl group.

The unsubstituted aryl group in Formula (1) may be a C6-C30 aromatic carbon ring that may include at least one ring, where the ring may be attached or fused by a pendent method. According to an embodiment of the present invention, examples of the aryl group may include, but are not limited to, phenyl, naphthyl, tetrahydronaphthyl, and the like. Moreover, at least one hydrogen atom of the aryl group may be substituted with the same substituent as in the above-described alkyl group.

In an additional embodiment, specific examples of the unsubstituted aryloxy group in the Formula (1) may include phenyloxy, naphthyloxy, diphenyloxy, and the like. Likewise, at least one hydrogen atom of the aryloxy group may be substituted with the same substituent as in the above-described alkyl group.

In a further embodiment, the unsubstituted arylalkyl group in Formula (1) may be the above-defined aryl group, where some hydrogen atoms are substituted with a lower alkyl, such as, methyl, ethyl, propryl, and the like. In an exemplary embodiment, an arylalkyl group may include, but is not limited to, benzyl, phenylethyl, and the like. Furthermore, at least one hydrogen atom of the arylalkyl group may be substituted with the same substituent as in the above-described alkyl group.

According to an embodiment of the present invention, the unsubstituted heteroaryl group in Formula (1) may be a monovalent monocylclic or divalent bicyclic aromatic organic group having about 2 to about 30 carbon atoms, containing one, two or three heteroatoms such as N, O, P, and S and may have carbon atoms as the remaining ring atoms. The heteroaryl group may include, but is not limited to, thienyl, pyridyl, furyl, and the like. Moreoever, at least one hydrogen atom in the heteroaryl group may be substituted with the same substituent as in the above-described alkyl group.

The unsubstituted heteroaryloxy group in Formula (1) may be the above-defined heteroaryl group with oxygen bonded thereto. Examples of such a heteroaryloxy group may include benzyloxy, phenylethyloxy, and the like. Additionally, at least one hydrogen atom of the heteroaryloxy group may be substituted with the same substituent as in the above-described alkyl group.

In a particular embodiment, examples of the unsubstituted arylalkyloxy group in Formula (1) may include a benzyloxy group, and the like, and where at least one hydrogen atom of the arylalkyloxy group may be substituted with the same substituent as in the above-described alkyl group.

The unsubstituted heteroarylalkyl group in Formula (1) may be the above-defined heteroaryl group, where some hydrogen atoms may be substituted with alkyl groups. Furthermore, at least one hydrogen atom of the heteroarylalkyl group may be substituted with the same substituent as in the above-described alkyl group.

Examples of the unsubstituted cycloalkyl group include cyclohexyl, cyclopentyl, and the like, where at least one hydrogen atom of the cycloalkyl group may be substituted with the same substituent as in the above-described alkyl group.

According to an embodiment of the present invention, a method for preparing an organic EL device adopting an organic layer using the pyrazole based bidentate ligand metal compound of the Formula (1) will now be described.

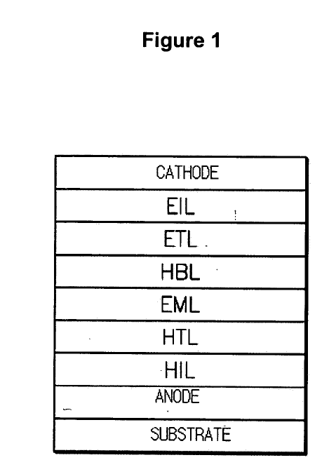

Referring to FIG. 1, which is a cross-sectional view of a general organic EL device, an anode is initially coated onto a substrate. In one embodiment, any substrate which is used in a conventional organic EL device may be used, such as a glass substrate or a transparent plastic substrate which is waterproof and has good transparency, surface softness, and capable of being easily handled may be used. The anode may be composed of, for example, indium tin oxide (ITO), indium zinc oxide (IZO), tin oxide (SnO₂), or zinc oxide (ZnO), which is transparent and has good conductivity.

Subsequently, a hole injection layer (HIL) may be deposited onto the anode by any method known on the art, such as thermal vapor deposition or by spin coating. The HIL may be composed of CuPc or a Starburst type amine, such as, TCTA, m-MTDATA, or m-MTDAPB, as illustrated below.

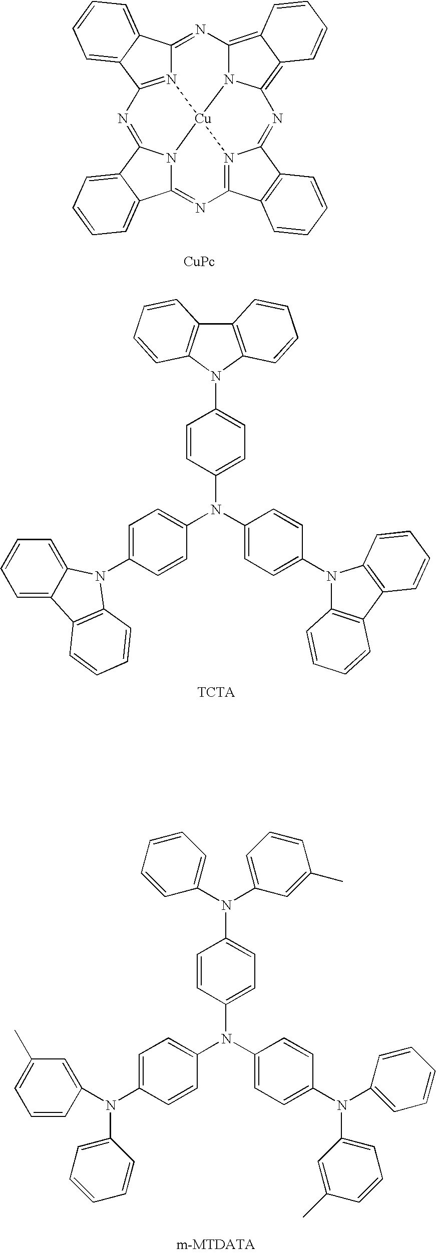

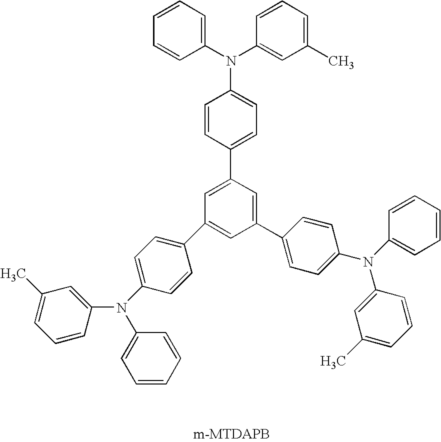

Then, a hole transport layer (HTL) may be deposited onto the HIL via thermal vapor deposition or by spin coating, for example. The HTL may be composed of N,N′-bis(3-methylphenyl)-N,N′-diphenyl-(1,1-biphenyl)-4,4′-diamine (TPD), N,N′-di(naphthalen-1-yl)-N,N′-diphenyl benzidine (α-NPD), or the like. Subsequently, an emissive layer (EML) may be formed on the HTL. In one embodiment, the EML may be composed of a carbazole containing compound of Formula (1), and in particular, one of the compounds of Formulas (2) through (8) may be used alone or as a light emitting host. When the compound of Formula (1) is used as the light emitting host, IDE102 or IDE105 available from Idemitsu may be used as the fluorescent dopant and Ir(ppy)₃ (phenylpyridine) (green), (4,6-F2ppy)₂Irpic (Adachi et al., supra), platinum (II) octaethylporphyrin (PtOEP), or TBE002 may be used as the phosphorescent dopant. A method of forming an EML may vary depending on the composition of the EML and, for example, thermal vapor deposition may be used.

According to an embodiment, the concentration of the dopant may be in the range of about 0.1 parts to about 30 parts by weight. In particular the dopant may be in the range of about 0.5 parts to about 12 parts by weight, based on 100 parts by weight of the EML, i.e., the total 100 parts by weight of the compound of Formula (1) as a host and the dopant. Specifically, when the concentration of the dopant is less than about 0.1 parts by weight, the dopant has a trivial effect. Alternatively, when the concentration of the dopant is greater than about 30 parts by weight, an undesirabe concentration quenching occurs in both the phosphorescence and the fluorescence.

In a further embodiment, an electron transport layer (ETL) may be subsequently formed on the EML using thermal vapor deposition or spin coating. The ETL may be composed of Alq3, as depicted below. 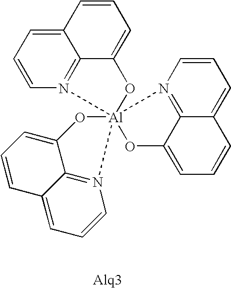
Additionally, in order to prevent a triplet exciton or a hole from diffusing into the ETL when the phosphorescent dopant is used in the EML, a hole blocking material may be formed by thermal vapor deposition on the EML to form a hole blocking layer. In particular, the hole blocking material may have an electron transporting ability and a higher ionization potential than the material used for forming an EML. The hole blocking material, for example, may be Balq, BCP, and the like, as illustrated below.

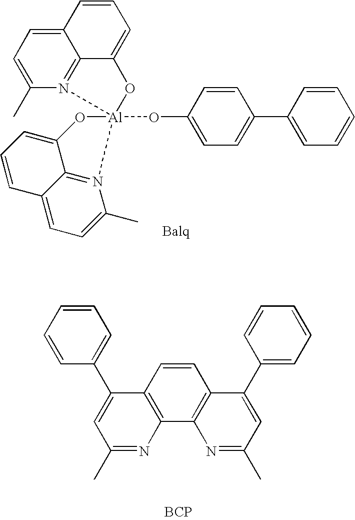

In an additional embodiment, an electron injection layer (EIL) may be selectively laminated on the ETL. The EIL may be composed of LiF, NaCl, CsF, Li₂O, or BaO, for example. Subsequently, a metal may be formed on the EIL via thermal vapor deposition, for example, thereby completing the organic EL device. The metal for forming the cathode may be Li, Mg, Al, Al—Li, Ca, Mg—In, or Mg—Ag, for example. Additionally, a transmittance type cathode composed of ITO or IZO may be used to form the front surface of the light emitting device.

The organic EL device according to an embodiment of the present invention may include one or two intermediate layers in addition to the anode, the HIL, the HTL, the EML, the HBL, the ETL, the EIL, and the cathode.

The present invention will be described in greater detail with reference to the following examples. The following examples are for illustrative purposes and are not intended to limit the scope of the invention.

### EXAMPLES

### Specific Example 1

### Synthesis of 2-Lithioanisole (A)

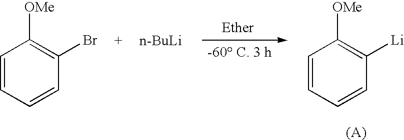

4.84 g (0.02588 mole) of 2-bromoanisole and 6.63 g (0.1035 mole) of n-butyl lithium were added to 40 ml of ether and reacted for three hours at −60° C. to obtain 2.85 g (0.02588 mole) of 2-lithioanisole.

### Specific Example 2

### Synthesis of 2-Methoxy phenylboronic acid (B)

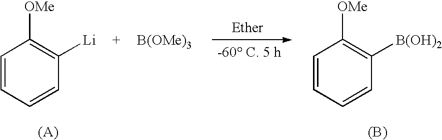

2.85 g (0.02588 mole) of 2-lithioanisole obtained from Example 1 and 2.69 g (0.02588 mole) of trimethoxyborane were added to 40 ml of ether and reacted for five hours at −60° C. to obtain 3.2 g (0.02104 mole) of 2-methoxy phenylboronic acid.

### Specific Example 3

### Synthesis of 1,3,5-tri(2-methoxyphenyl)benzene (C)

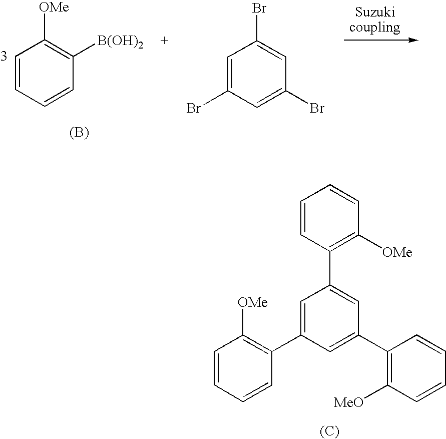

3.2 g (0.02104 mole) of 2-methoxy phenylboronic acid obtained from Example 2 was reacted with 2.2 g (0.007012 mole) of 1,3,5-tribromobenzene through a Suzuki coupling reaction to obtain 2.72 g (0.006858 mole) of 1,3,5-tri(2-methoxyphenyl)benzene.

### Specific Example 4

### Synthesis of 1,3,5-tri(2-methoxy-5-bromophenyl)benzene (D)

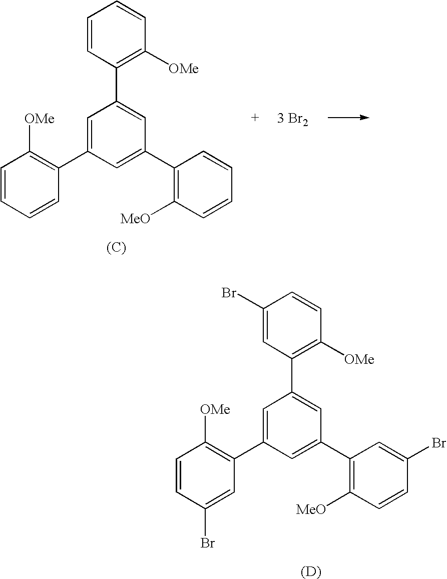

2.72 g (0.006858 mole) of 1,3,5-tri(2-methoxyphenyl)benzene obtained from Example 3 and 3 equivalents of Br₂ were added to 40 ml of CCl₄ and reacted for 12 hours at 20° C. to obtain 3.86 g (0.00609 mole) of 1,3,5-tri(2-methoxy-5-bromophenyl) benzene.

### Specific Example 5

### Synthesis of the Compound of Formula (2)

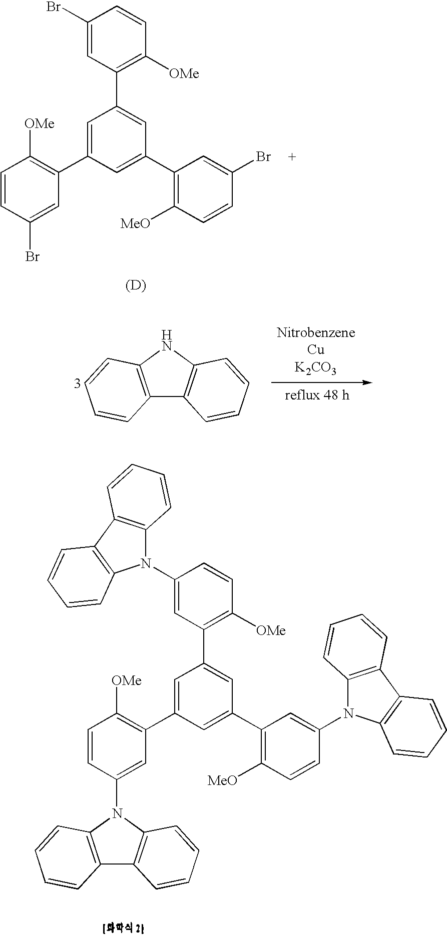

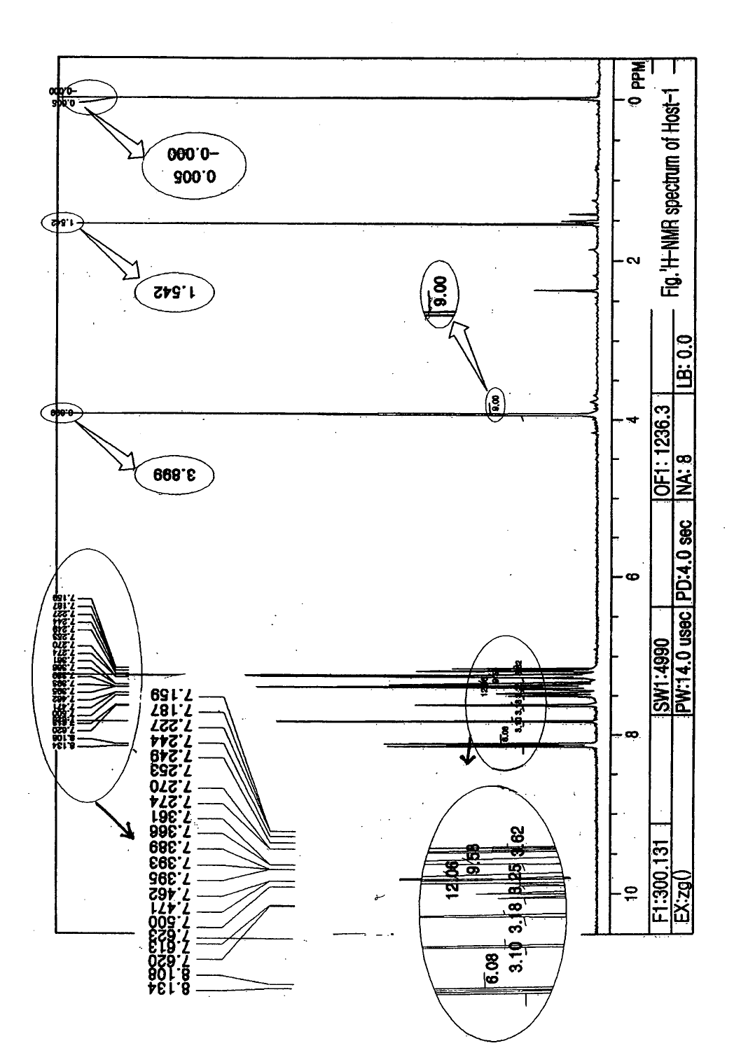

3.86 g (0.00609 mole) of 1,3,5-tri(2-methoxy-5-bromophenyl)benzene and 3 equivalents of carbazole were reacted with 20 ml of nitrobenzene, 0.5 g of copper, and 3.79 g of K₂CO₃ for 48 hours to obtain 1 g (0.001121 mole) of the compound of Formula (2). The structure of the compound of Formula (2) was identified from an NMR spectrum shown in FIG. 2.

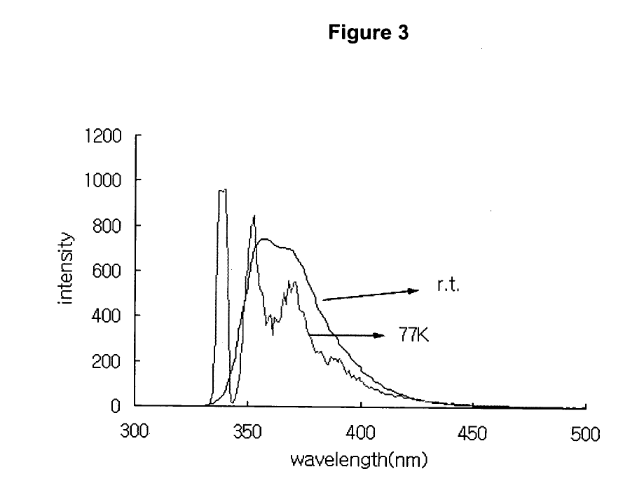

Further, photoluminescence (PL) of the compound of Formula (2) was investigated at room temperature and at 77K, respectively, and the results are shown in FIG. 3. Referring to FIG. 3, since it is easier to change the structure of the compound in an excited state at room temperature than at 77K, the PL spectrum is slightly red-shifted at room temperature.

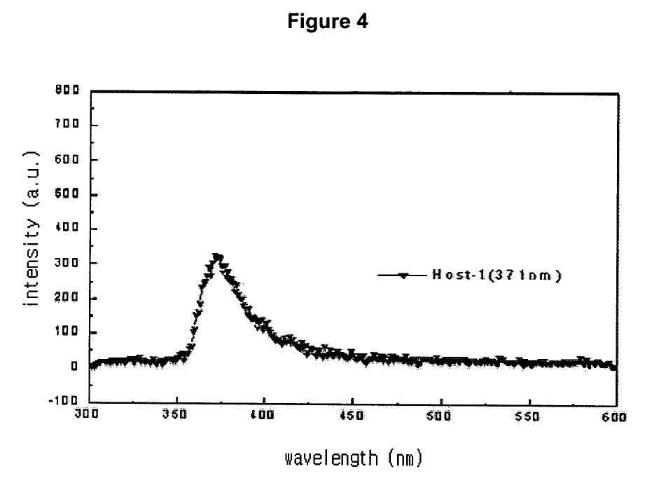

The compound of Formula (2) obtained from Example 4 was diluted in CH₂Cl₂ to 10⁻⁴ mM solution to investigate PL characteristic at 333 nm, and the result is shown in FIG. 4. Referring to FIG. 4, the compound of Formula (2) has a maximum luminescence at about 370 nm.

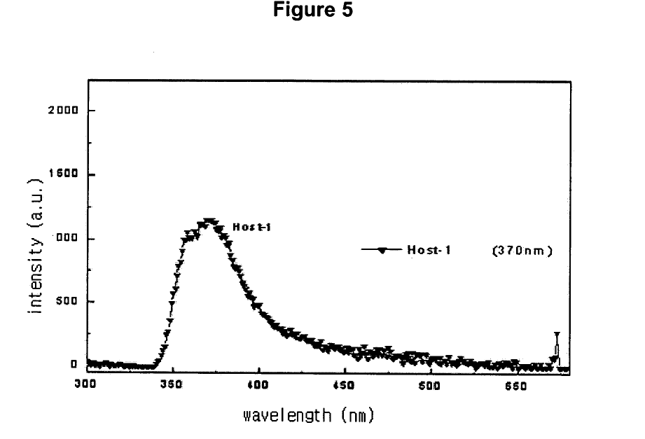

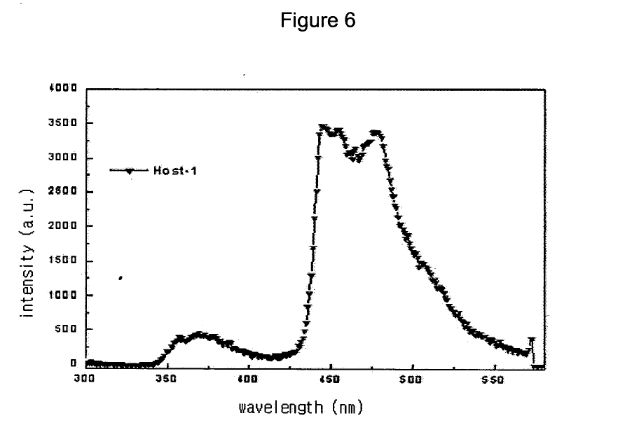

The compound of Formula (2) and polymethylmethacrylate (PMMA) polymer at a ratio of 90:10 by weight were dissolved in chloroform and then spin coated on a glass substrate to form a thin film. Then, PL characteristics of the thin film were measured and the maximum luminescence was observed at 370 nm (FIG. 5). Further, the compound of Formula (2) (90 part by weight) and TEB002 dopant (10 parts by weight) were dissolved in chloroform and spin coated on a glass substrate to form a thin film. Then, PL characteristics of the thin film were measured and the maximum luminescence was observed at 450 nm (FIG. 6).

### Specific Example 6

IDE406 (Idemitsu, Japan) was vacuum deposited on 10 Ω/cm² ITO substrate (Corning, Corning, N.Y.) as an anode to form an HIL having a thickness of 600 Å. Then, IDE330 (Idemitsu, Japan) was vacuum deposited to a thickness of 300 Å on the HIL to form an HTL. A mixture of the compound of Formula (2) and TEB002 dopant at a ratio of 90:10 by weight was vacuum deposited on the HTL to form an EML having a thickness of 300 Å.

Then, Balq was vacuum deposited on the EML to form an HBL. Then, Alq3 was vacuum deposited on the HBL to form an ETL having a thickness of 300 Å. LiF and Al were sequentially vacuum deposited on the ETL to a thicknesses of 10 Å and 3000 Å, respectively, to form a cathode, thereby completing an organic EL device.

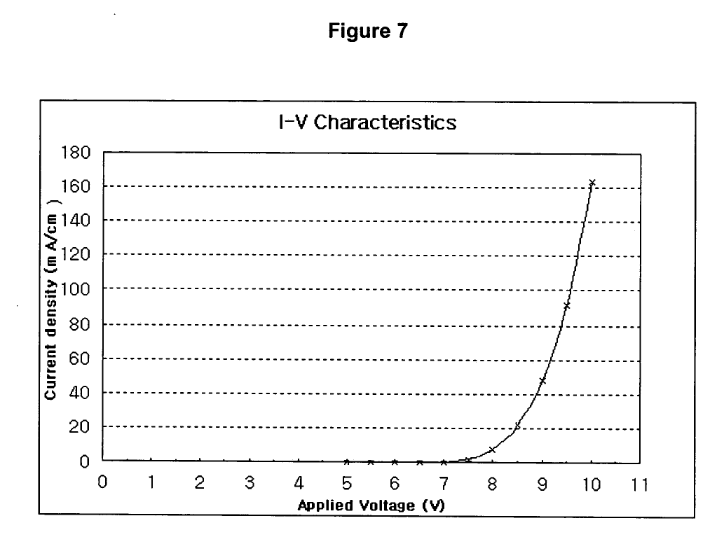

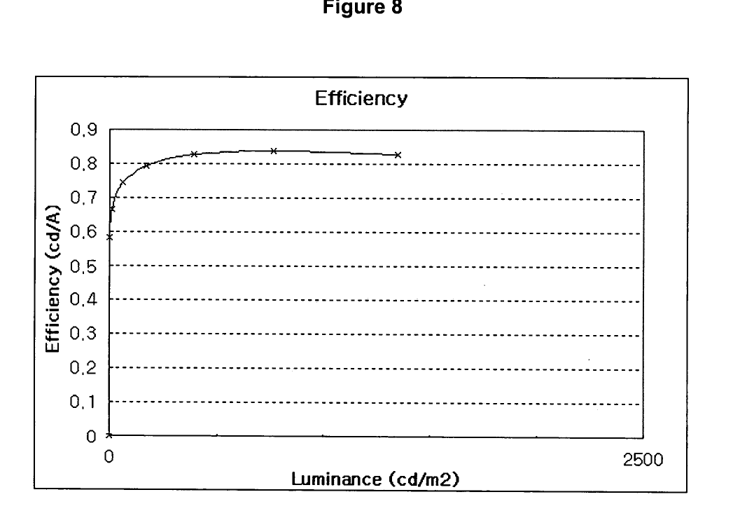

A graph showing the current density of the organic EL device prepared according to Example 1 with respective to voltage is shown in FIG. 7. Referring to FIG. 7, the organic EL device starts driving at about 8V. Efficiency with respect to brightness was investigated in the organic EL device prepared according to Example 1 and the result is shown in FIG. 8. Referring to FIG. 8, the organic EL device has an efficiency of about 0.83 cd/A and only a low efficiency decrease at high luminance.

As described above, carbazole containing compounds according to embodiments of the present invention may compare a blue phosphorescent host material and may have a large triplet energy band gap compared to CBP, thus effectively achieving energy transferral to blue dopants. When adopting an organic layer formed using the carbazole containing compounds, an organic EL device having a high efficiency and improved color purity and power consumption characteristics can be prepared. The carbazole containing compounds according to embodiments of the present invention are useful for forming an organic layer, such as an emissive layer of an organic EL device.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A carbazole containing compound, comprising Formula (1): 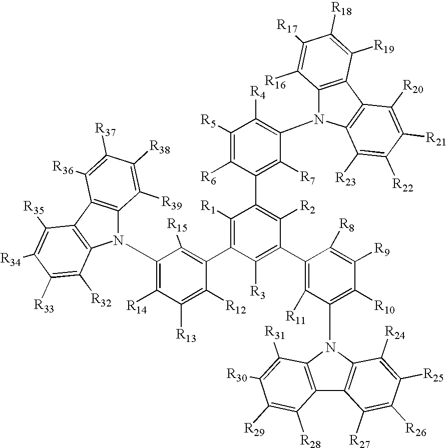
where each of R₁ through R₃₉ are independently selected from the group consisting of a hydrogen atom, a cyano group, a hydroxy group, a thiol group, a halogen atom, a substituted or unsubstituted C1-C30 alkyl group, a substituted or unsubstituted C1-C30 alkoxy group, a substituted or unsubstituted C2-C30 alkenyl group, a substituted or unsubstituted C2-C30 alkylcarbonyl group, a substituted or unsubstituted C6-C30 aryl group, a substituted or unsubstituted C7-C30 arylcarbonyl group, a substituted or unsubstituted C6-C30 arylalkyl group, a substituted or unsubstituted C6-C30 aryloxy group, a substituted or unsubstituted C2-C30 heteroaryl group, a substituted or unsubstituted C2-C30 heteroarylalkyl group, a substituted or unsubstituted C2-C30 heteroaryloxy group, a substituted or unsubstituted C2-C30 heteroarylalkyloxy group, a substituted or unsubstituted C5-C30 cycloalkyl group, a substituted or unsubstituted C2-C30 heterocycloalkyl group, a substituted or unsubstituted C1-C30 alkylthio group, —Si(R′)(R″)(R′″) (where of R′, R″, and R′″ is hydrogen or a C1-C30 alkyl group) and —N(R′)(R″) (where each of R′ and R″ is hydrogen or a C1-C30 alkyl group) or where two or more adjacent groups selected from R₄-R₆, R₈-R₁₀, R₁₂-R₁₄, R₁₆-R₁₉, R₂₀-R₂₃, R₂₄-R₂₇, R₂₈-R₃₁, R₃₂-R₃₅, and where R₃₆-R₃₉ are linked to each other to form an unsaturated or saturated ring.

2. The carbazole containing compound of claim 1, wherein the compound is selected from the group consisting of a compound of Formula (1) where each of R₆, R₈, and R₁₂ is a methoxy group, and each of R₁-R₅, R₇, R₉, R₁₀, R₁₁, and R₁₃-R₃₉ is hydrogen, the compound of Formula (1) where each of R₆, R₈, and R₁₂ is a methyl group, and where each of R₁-R₅, R₇, R₉, R₁₀, R₁₁, and where R₁₃-R₃₉ is hydrogen, the compound of Formula (1) where each of R₄, R₇, R₁₀, R₁₁, R₁₄, and R₁₅ is a methyl group, and where each of R₁-R₃, R₅, R₆, R₈, R₉, R₁₂, R₁₃ and where R₁₆-R₃₉ is hydrogen, the compound of Formula (1) where each of R₄, R₆, R₈, R₁₀, R₁₂, and R₁₄ is a methyl group, and where each of R₁-R₃, R₅, R₇, R₉, R₁₁, R₁₃, and where R₁₅-R₃₉ is hydrogen, the compound of Formula (1) where each of R₁-R₃₉ is hydrogen, and the compound of Formula (1) where each of R₄, R₆, R₈, R₁₀, R₁₂, R₁₄, R₁₈, R₂₁, R₂₆, R₂₉, R₃₄, and where R₃₇ is a methyl group, and where each of R₁-R₃, R₅, R₇, R₉, R₁₁, R₁₃, R₁₅-R₁₇, R₁₉, R₂₀, R₂₂-R₂₅, R₂₇, R₂₈, R₃₀-R₃₃, R₃₅, R₃₆, R₃₈, and where R₃₉ is hydrogen.

3. The carbazole containing compound of claim 1, where the compound comprises a compound of Formula (8):

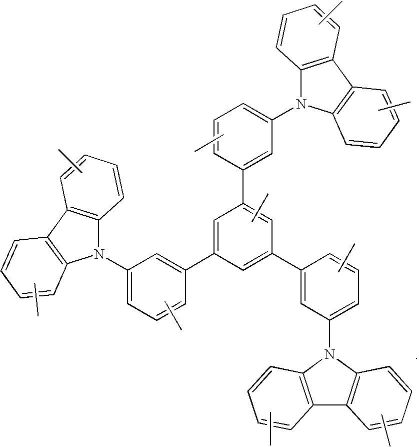

4. An organic electroluminescent display device comprising an organic layer interposed between a pair of electrodes, wherein the organic layer comprises the carbazole containing compound of Formula (1): 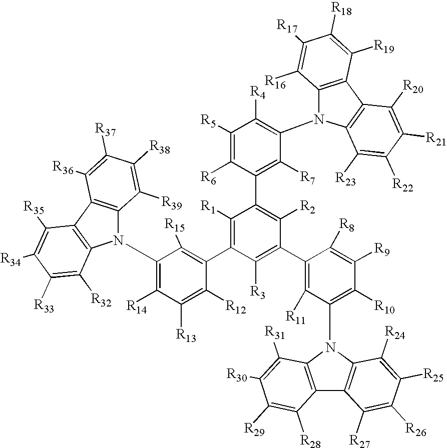
where each of R₁ through R₃₉ are independently selected from the group consisting of a hydrogen atom, a cyano group, a hydroxy group, a thiol group, a halogen atom, a substituted or unsubstituted C1-C30 alkyl group, a substituted or unsubstituted C1-C30 alkoxy group, a substituted or unsubstituted C2-C30 alkenyl group, a substituted or unsubstituted C2-C30 alkylcarbonyl group, a substituted or unsubstituted C6-C30 aryl group, a substituted or unsubstituted C7-C30 arylcarbonyl group, a substituted or unsubstituted C6-C30 arylalkyl group, a substituted or unsubstituted C6-C30 aryloxy group, a substituted or unsubstituted C2-C30 heteroaryl group, a substituted or unsubstituted C2-C30 heteroarylalkyl group, a substituted or unsubstituted C2-C30 heteroaryloxy group, a substituted or unsubstituted C2-C30 heteroarylalkyloxy group, a substituted or unsubstituted C5-C30 cycloalkyl group, a substituted or unsubstituted C2-C30 heterocycloalkyl group, a substituted or unsubstituted C1-C30 alkylthio group, —Si(R′)(R″)(R′″) (where of R′, R″, and R′″ is hydrogen or a C1-C30 alkyl group) and —N(R′)(R″) (where each of R′ and R″ is hydrogen or a C1-C30 alkyl group) or where two or more adjacent groups selected from R₄-R₆, R₈-R₁₀, R₁₂-R₁₄, R₁₆-R₁₉, R₂₀-R₂₃, R₂₄-R₂₇, R₂₈-R₃₁, R₃₂-R₃₅, and where R₃₆-R₃₉ are linked to each other to form an unsaturated or saturated ring.

5. The organic electroluminescent display device of claim 4, wherein the organic layer is a light emitting layer.

6. The organic electroluminescent display device of claim 4, wherein the light emitting layer further includes a phosphorescent or a fluorescent dopant capable of producing visible light.

7. The organic electroluminescent display device of claim 4, wherein the carbazole containing compound is selected from the group consisting of the compound of Formula (1) where each of R₆, R₈, and R₁₂ is a methoxy group, and each of R₁-R₅, R₇, R₉, R₁₀, R₁₁, and where R₁₃-R₃₉ is hydrogen, the compound of Formula (1) where each of R₆, R₈, and R₁₂ is a methyl group, and where each of R₁-R₅, R₇, R₉, R₁₀, R₁₁, and R₁₃-R₃₉ is hydrogen, the compound of Formula (1) where each of R₄, R₇, R₁₀, R₁₁, R₁₄, and where R₁₅ is a methyl group, and where each of R₁-R₃, R₅, R₆, R₈, R₉, R₁₂, R₁₃ and where R₁₆-R₃₉ is hydrogen, the compound of Formula (1) where each R₄, R₆, R₈, R₁₀, R₁₂, and where R₁₄ is a methyl group, and where each of R₁-R₃, R₅, R₇, R₉, R₁₁, R₁₃, and R₁₅-R₃₉ is hydrogen, the compound of Formula (1) where each of R₁-R₃₉ is hydrogen, and the compound of Formula (1) where each of R₄, R₆, R₈, R₁₀, R₁₂, R₁₄, R₁₈, R₂₁, R₂₆, R₂₉, R₃₄, and where R₃₇ is a methyl group, and each of R₁-R₃, R₅, R₇, R₉, R₁₁, R₁₃, R₁₅-R₁₇, R₁₉, R₂₀, R₂₂-R₂₅, R₂₇, R₂₈, R₃₀-R₃₃, R₃₅, R₃₆, R₃₈, and where R₃₉ is hydrogen.

8. The organic electroluminescent display device of claim 4, wherein the carbazole containing compound comprises a compound of Formula (8): [chemical structure]
